(19) United States
(12) Patent Application Publication
(10) Pub. No.: US 2023/0355528 A1
(43) Pub. Date: Nov. 9, 2023

(54) LIPID NANODISC FORMATION BY POLYSACCHARIDES MODIFIED WITH HYDROPHOBIC GROUPS

(71) Applicant: THE REGENTS OF THE UNIVERSITY OF MICHIGAN, Ann Arbor, MI (US)

(72) Inventors: Ayyalusamy Ramamoorthy, Ann Arbor, MI (US); Thirupathi Ravula, Ann Brbor, MI (US)

(21) Appl. No.: 18/029,064

(22) PCT Filed: Oct. 20, 2021
(86) PCT No.: PCT/US21/55749

§ 371 Date: Mar. 28, 2023

Related U.S. Application Data
(60) Provisional application No. 63/173,751, filed on Apr. 12, 2021.
(60) Provisional application No. 63/198,640, filed on Oct. 30, 2020.

(51) Int. Cl. A61K 9/127; A61K 47/36; A61K 39/39; G01N 33/68

(52) U.S. Cl. CPC A61K 9/1274; A61K 9/1277; A61K 47/36; A61K 9/1272; A61K 39/39; G01N 33/6842; A61K 2039/55555

(57) ABSTRACT

The disclosure generally relates to lipid nanodiscs, in particular to lipid nanodiscs formed from polysaccharides. A lipid nanodisc according to the disclosure includes a lipid bilayer having a first hydrophilic face and a second hydrophilic face opposing the first hydrophilic face, and a hydrophobic edge between the opposing hydrophilic faces, and a polysaccharide encircling the hydrophobic edge of the lipid bilayer. The polysaccharide is modified with a hydrophobic group. Methods of making and characterizing the lipid nanodiscs are also disclosed.

### STATEMENT OF GOVERNMENT SUPPORT

This invention was made with government support under GM084018 and AG048934 awarded by the National Institutes of Health. The government has certain rights in the invention.

### FIELD OF THE DISCLOSURE

The disclosure relates generally to lipid nanodiscs. In particular, the disclosure relates to lipid nanodiscs formed with polysaccharides that are modified with hydrophobic groups.

### BACKGROUND

Membrane proteins play many crucial roles in the cellular function that are related to many diseases including cancer, heart disease, and infection diseases. More than 60% of the drugs available in the current market target membrane proteins such as GPCRs. To fully understand the function of membrane proteins, it is important to obtain their atomic-resolution structure, dynamics, and function. Determination of the structure and function of membrane proteins is a challenge due to the difficulty of developing methods of extracting membrane proteins from their native environment, while preserving the correct conformation of the protein in isolation from its native environment. Traditional protocols involve extracting membrane proteins from their native environment using detergents and then including the proteins in a model bilayer system. Unfortunately, the use of detergents leads to issues such as protein inactivation and sample aggregation.

In order to avoid the use of detergents, methods for the isolation, purification, and characterization of membrane proteins have been developed which reconstitute membrane proteins in nanodiscs. Nanodiscs are disc-shaped patches of lipid bilayers surrounded by an amphiphilic belt. Amphiphilic belts that have been used in preparing nanodiscs include different sized membrane scaffold proteins, peptides, and polymers. Membrane scaffold protein-based nanodiscs are good mimics of the membrane; however, the reconstitution of the membrane proteins still require the use of detergents. Additionally, protein-based nanodiscs are restricted to a narrow range of size, difficult to prepare, and expensive to produce. Peptide-based nanodiscs are also limited by several disadvantages, including stability issues, interference from the peptides in biophysical measurements, and are expensive to produce. Similarly, copolymer-based nanodiscs are limited by disadvantages including restricted size range, their non-tolerance in the presence of divalent metal ions and different pH, and are expensive to produce. Thus, a need exists for nanodiscs that can address these difficulties.

Further, many useful drugs and vaccines are hydrophobic and/or have low solubility in aqueous environments, such as physiological environments. The lack of solubility of such drugs and vaccines decreases the bioavailability of such drugs and vaccines. In order to increase the solubility and resulting bioavailability, solubilizing carriers or adjuvants need to be included in the formulation that is administered. Thus a need exists for carriers and adjuvants that can increase the solubility of hydrophobic and/or low solubility drugs and vaccines.

### SUMMARY

Provided herein are lipid nanodiscs, comprising a lipid bilayer comprising a first hydrophilic face and a second hydrophilic face opposing the first hydrophilic face, and a hydrophobic edge between the opposing hydrophilic faces, and a polysaccharide encircling the hydrophobic edge of the lipid bilayer, wherein the polysaccharide is modified with a hydrophobic group.

In embodiments, the polysaccharide comprises a linear polysaccharide having a degree of polymerization of about 100 or less. In embodiments, the polysaccharide is one or more selected from the group of inulin, cellulose, amylose, and any derivative of the foregoing.

In embodiments, the hydrophobic group is C₁₋₂₀ alkyl, C₁₋₂₀ haloalkyl, C₂₋₂₀alkenyl, C₂₋₂₀ alkynyl, C₃₋₂₀ cycloalkyl, C₆₋₂₀ aryl, or C₁₋₂₀ heteroalkyl having 1-3 backbone heteroatoms selected from N, O, and S. In embodiments, the hydrophobic group is substituted with one or more of C₁₋₆ alkyl, C₃₋₁₀cycloalkyl, and C₆₋₁₀aryl. In embodiments, the hydrophobic group is one or more selected from the group of n-hexyl, n-pentyl, n-butyl, phenyl, benzyl, biphenyl, pyrene, and adamantane. In embodiments, the polysaccharide is inulin modified with one or more selected from the group of n-hexyl, n-pentyl, n-butyl, phenyl, benzyl, and biphenyl.

In embodiments, the hydrophobic group is bound to the polysaccharide (e.g., backbone ring unit thereof) via a linking group selected from the group consisting of an ether group, an ester group, and an amide group.

In embodiments, the polysaccharide is free of amine. In embodiments, about 5% to about 50% of pendant oxygen atoms on the polysaccharide are modified with a hydrophobic group.

In embodiments, the polysaccharide is free of charge at a pH of about 2 to about 12.

In embodiments, the polysaccharide has an average degree of polymerization of about 5 to about 100.

In embodiments, the polysaccharide has a weight-average molecular weight (M_{w}) of about 1 kg/mol to about 50 kg/mol.

In embodiments, the lipid comprises at least one of phosphatidylethanolamines, phosphatidylcholines, phosphatidylglycerols, phosphatidylserines, cholesterols, sphingomyelin, gangliosides, lipopolysaccharides, phosphatidylinositols, and derivatives of the foregoing. In embodiments, the lipid comprises dipalmitoylphosphatidylcholine (DMPC).

In embodiments, the lipid comprises a natural cell membrane extract.

In embodiments, the lipid and the polysaccharide are present in a weight ratio of about 1:0.1 to about 1:20.

In embodiments, the nanodisc has a diameter in a range of about 6 nm to about 100 nm.

In embodiments, the nanodisc is stable at a pH level ranging from about 2 to about 9. In embodiments, the nanodisc is stable in the presence of up to about 150 mM of a divalent metal ion. In embodiments, the nanodisc is stable in the presence of 100 mM of Ca²⁺.

In embodiments, the lipid nanodisc further comprises a membrane protein spanning across the lipid bilayer from the first hydrophilic face to the second hydrophilic face. In embodiments, the lipid nanodisc is characterized in that when a magnetic field is applied, the nanodisc aligns with the magnetic field. In embodiments, the lipid nanodisc is characterized by a solubilization efficiency of at least 50%.

Also provided are methods of characterizing a membrane protein, the method comprising contacting the lipid nanodisc of any one of the preceding claims with a membrane protein to form a membrane protein-nanodisc comprising the membrane protein spanning across the lipid bilayer from the first hydrophilic face to the second hydrophilic face, and characterizing the lipid nanodisc comprising the membrane protein.

In embodiments, the contacting comprises admixing the lipid nanodisc and membrane protein in solution. In embodiments, the solution is substantially free of a detergent.

In embodiments, the lipid comprises a natural cell membrane extract.

In embodiments, characterizing comprises at least one of structural characterization and functional characterization, the characterization comprising performing at least one of solution nuclear magnetic resonance (NMR), solid state NMR, circular dichroism, electron paramagnetic resonance (EPR), Fourier transform infrared spectroscopy (FTIR), resonance Raman spectroscopy, ultraviolet-visible spectroscopy (UV/vis), cryo-electron microscopy (cryo-EM), surface plasmon Raman spectroscopy, sum frequency generation (SFG), fluorescence, small angle x-ray scattering (SAXS), scanning electron microscopy (SEM), atomic force microscopy (AFM), and an enzymatic assay.

Also provided are methods of preparing the lipid nanodisc of the disclosure, the methods comprising contacting the lipid and the polysaccharide to form the lipid nanodisc.

In embodiments, the method further comprises preparing an aqueous solution of the polysaccharide prior to contacting the lipid with the polysaccharide, and the solution is substantially free of a detergent.

In embodiments, the pH of the solution is in a range of about 0 to 6. In embodiments, the pH of the solution is in a range of about 2.5 to 10. In embodiments, the pH of the solution is in a range of about 2 to about 10.

In embodiments, the method further comprises preparing a lipid dispersion prior to contacting the lipid with the polysaccharide. In embodiments, the lipid dispersion is substantially free of a detergent.

In embodiments, the lipid is provided as a multilamellar vesicle. In embodiments, the lipid comprises a natural cell membrane extract.

In embodiments, the contacting comprises admixing the lipid and the polysaccharide in solution. In embodiments, the solution is substantially free of a detergent.

Further aspects and advantages will be apparent to those of ordinary skill in the art from a review of the following detailed descriptions. While the compositions and methods are susceptible of embodiments in various forms, the description hereafter includes specific embodiments with the understanding that the disclosure is illustrative, and is not intended to limit the invention to the specific embodiments described herein.

### BRIEF DESCRIPTION OF THE DRAWINGS

FIG. 1 shows a simplified schematic representation of lipid nanodisc formation.

FIG. 2 is a graph of the static light scattering profiles of lipid nanodiscs according to the disclosure with polysaccharides modified with various hydrophobic groups.

FIG. 3 is a transmission electron microscopy (TEM) image of lipid nanodiscs according to the disclosure (scale=20 nm).

FIG. 4A is an image of lipid nanodiscs according to the disclosure in a solution having a pH of 8.5.

FIG. 4B is an image of lipid nanodiscs according to the disclosure in a solution having a pH of 7.4.

FIG. 4C is an image of lipid nanodiscs according to the disclosure in a solution having a pH of 5.8.

FIG. 4D is an image of lipid nanodiscs according to the disclosure in a solution having a pH of 4.8.

FIG. 4E is an image of lipid nanodiscs according to the disclosure in a solution having a pH of 3.8.

FIG. 4F is an image of lipid nanodiscs according to the disclosure in a solution having a pH of 3.3.

FIG. 4G is an image of lipid nanodiscs according to the disclosure in a solution having a pH of 2.7.

FIG. 4H is an image of lipid nanodiscs according to the disclosure in a solution having a pH of 2.1.

FIG. 5A is an image of a DMPC liposomes, having no modified polysaccharide, in solution.

FIG. 5B is an image of lipid nanodiscs according to the disclosure in a solution having a [Ca²⁺] of 0 mM.

FIG. 5C is an image of lipid nanodiscs according to the disclosure in a solution having a [Ca²⁺] of 2 mM.

FIG. 5D is an image of lipid nanodiscs according to the disclosure in a solution having a [Ca²⁺] of 5 mM.

FIG. 5E is an image of lipid nanodiscs according to the disclosure in a solution having a [Ca²⁺] of 10 mM.

FIG. 5F is an image of lipid nanodiscs according to the disclosure in a solution having a [Ca²⁺] of 50 mM.

FIG. 5G is an image of lipid nanodiscs according to the disclosure in a solution having a [Ca²⁺] of 75 mM.

FIG. 5H is an image of lipid nanodiscs according to the disclosure in a solution having a [Ca²⁺] of 100 mM.

FIG. 6A is a ³¹P NMR of lipid nanodiscs according to the disclosure at a temperature of 315 K.

FIG. 6B is a ³¹P NMR of lipid nanodiscs according to the disclosure at a temperature of 312 K.

FIG. 6C is a ³¹P NMR of lipid nanodiscs according to the disclosure at a temperature of 310 K.

FIG. 6D is a ³¹P NMR of lipid nanodiscs according to the disclosure at a temperature of 308 K.

FIG. 6E is a ³¹P NMR of lipid nanodiscs according to the disclosure at a temperature of 305 K.

FIG. 6F is a ³¹P NMR of lipid nanodiscs according to the disclosure at a temperature of 302 K.

FIG. 6G is a ³¹P NMR of lipid nanodiscs according to the disclosure at a temperature of 300 K.

FIG. 6H is a ³¹P NMR of lipid nanodiscs according to the disclosure at a temperature of 298 K.

FIG. 6I is a ³¹P NMR of lipid nanodiscs according to the disclosure at a temperature of 295 K.

FIG. 6J is a ³¹P NMR of lipid nanodiscs according to the disclosure at a temperature of 293 K.

FIG. 7 is a bar graph of the solubilization efficiency of E. coli cell membranes by lipid nanodiscs.

### DETAILED DESCRIPTION

Provided herein are polysaccharide-based lipid nanodiscs and methods of making and using same. The polysaccharide-based lipid nanodiscs disclosed herein include a polysaccharide that is modified with hydrophobic groups. As used herein, the terms “polysaccharide that is modified with hydrophobic groups” and “modified polysaccharide” are used interchangeably. In embodiments, the modified polysaccharide is free of charge. As used herein, the term “free of charge” or “charge-free” means that neither the polysaccharide as a whole nor any functional groups thereof carry a positive or a negative charge. The present inventors have found that presence of a charge on the amphiphilic belt can limit the membrane proteins that can be studied to only those that possess the same charge. For example, a positively-charged polymer can only reconstitute a positively charged protein. Advantageously, the present inventors found that these limitations can be overcome by forming a lipid nanodisc that is charge-free using a charge-free, hydrophobic-modified polysaccharide polymer. These charge-free polymers, such as the modified polysaccharides of the disclosure, can be used to form lipid nanodiscs that are stable at a wide range of pH levels and in the presence of ions, and that can be used to directly extract membrane proteins from cells.

The modified polysaccharides described herein provide one or more advantages, for example, extracting membrane proteins without the use of low-molecular weight detergents, forming nanodiscs with native lipid bilayers, solubilization of lipid bilayers, and forming nanodiscs over wide pH ranges and sizes. Additionally, the modified polysaccharides are advantageously stable for periods of at least 6 months, can be stored as powders, and do not require purification by high performance liquid chromatography. Furthermore, the lipid nanodiscs of the disclosure provide the unique advantage of enabling the application of various biophysical techniques typically employed in the structural study of membrane proteins, such as circular dichroism, UV/vis, and fluorescence spectroscopy, which may be otherwise unsuitable for lipid nanodiscs previously reported in the art.

### Modified Polysaccharides

The lipid nanodiscs include a polysaccharide modified with hydrophobic groups, for example via the hydroxy and/or amino groups that would otherwise be present in a native or unmodified polysaccharide. The modified polysaccharide can include any linear polysaccharide having a degree of polymerization of about 100 or less. The modified polysaccharide can have a have a degree of polymerization of about 100 or less, for example, about 100, 95, 90, 85, 80, 75, 70, 60, 50, 40, 30, 20, 10 or 5 or less. In embodiments, the modified polysaccharide has a degree of polymerization of about 5 to about 100, about 10 to about 100, about 15 to about 85, about 20 to about 80, or about 30 to about 60. The modified polysaccharides can be modified versions of polysaccharides including, but not limited to, inulin, cellulose, chitosan, amylose, derivatives of the foregoing, and combinations of the foregoing. In embodiments, the modified polysaccharide includes modified inulin or a derivative thereof. Inulins can include naturally occurring polysaccharides produced by various plants, and they can be classified as fructans or fructose polymers/oligomers, generally having a fructosyl backbone (linked by beta (2,1) bonds) and terminating glucosyl groups. In embodiments, the modified polysaccharide includes modified cellulose or a derivative thereof. In embodiments, the polysaccharide includes modified chitosan or a derivative thereof. In embodiments, the polysaccharide includes modified amylose or a derivative thereof. In embodiments, the polysaccharide includes one or more monosaccharide residues such as glucose, fructose, galactose, glucosamine (deacetylated and/or N-acetylated), etc., for example including linear blocks of one of the foregoing monosaccharide residues, optionally being terminated by a different residue. Derivatives of the polysaccharide can include esters and ethers of the polysaccharide. For example, suitable derivatives of cellulose can include carboxymethylcellulose (CMC), hydroxypropyl methylcellulose (HPMC), etc.

The polysaccharide is modified to include hydrophobic groups. For example, the native hydroxyl and/or amine functional groups of the polysaccharide can be modified to include a hydrophobic group. For example, the hydrophobic group can be bound to the polysaccharide (e.g., backbone ring unit thereof) via a linking group, such as an ether group, an ester group, and an amide group. In embodiments, hydroxyl functional groups can be modified with the hydrophobic groups of the disclosure to provide a hydrophobic ether or ester group (e.g., —OH to —OR or —OH to —O—C(O)—R, wherein R is the hydrophobic group). The amine functional groups can be modified with the hydrophobic groups of the disclosure to provide a hydrophobic amide group (e.g., —NH₂ to —NH—C(O)—R, wherein R is the hydrophobic group). In embodiments, about 5% to about 50% of hydroxyl functional groups of the polysaccharide are modified with a hydrophobic group, for example at least about 5, 10, 15, 20, 25, 30, or 35% and/or up to about 20, 25, 30, 35, 40, 45, or 50% of the hydroxyl functional groups of the polysaccharide are modified with a hydrophobic group, such as about 5% to about 50%, about 10% to about 45%, about 15% to about 40%, about 20% to about 50%, about 35% to about 40%, or about 20% to about 30%. In embodiments, the modified polysaccharide is free of amine. As used herein, “free of amine” mean that 100% of the amine functional groups are modified with a hydrophobic group. Namely, when the native polysaccharide contains amino groups, it is preferred that the corresponding modified polysaccharide has been functionalized at essentially all of the amino group locations so that there will be no net charge on the modified polysaccharide

The hydrophobic group can include C₁₋₂₀ alkyl, C₁₋₂₀ haloalkyl, C₂₋₂₀ alkenyl, C₂₋₂₀alkynyl, C₃₋₂₀ cycloalkyl, C₆₋₂₀ aryl, or C₁₋₂₀ heteroalkyl having 1-3 backbone heteroatoms selected from N, O, and S. The foregoing hydrophobic groups can be substituted or unsubstituted.

In embodiments, the hydrophobic group includes C₁₋₂₀ alkyl. As used herein, the term “alkyl” refers to straight chained and branched saturated hydrocarbon groups. The term Cn means the group has “n” carbon atoms. For example, C₆ alkyl refers to an alkyl group that has 6 carbon atoms. C₁₋₂₀ alkyl refers to an alkyl group having a number of carbon atoms encompassing the entire range (i.e., 1 to 20 carbon atoms), as well as all subgroups (e.g., 2-20, 3-17, 5-15, 5-20, 1-15, 6-10, 10-20, 1-10, 1, 2, 3, 4, 5, 6, 7, 8, 9, 10, 11, 12, 13, 14, 15, 16, 17, 18, 19 or 20 carbon atoms). Nonlimiting examples of alkyl groups include methyl, ethyl, n-propyl, isopropyl, n-butyl, sec-butyl (2-methylpropyl), tert-butyl (1,1-dimethylethyl), n-pentyl, n-hexyl, n-heptyl, n-octyl, n-nonyl, n-decyl, and n-eicosyl. Unless otherwise indicated, an alkyl group can be an unsubstituted alkyl group or a substituted alkyl group. In general, a methyl hydrophobic group may be too small to form the lipid nanodiscs of the disclosure, if used as the sole hydrophobic group. However, a methyl hydrophobic group can be used in combination with other hydrophobic groups, e.g., C₂₋₂₀ alkyl, C₁₋₂₀ haloalkyl, C₂₋₂₀ alkenyl, C₂₋₂₀ alkynyl, C₃₋₂₀cycloalkyl, C₆₋₂₀ aryl, or C₁₋₂₀ heteroalkyl having 1-3 backbone heteroatoms selected from N, O, and S.

In embodiments, the hydrophobic group includes C₁₋₂₀ haloalkyl, for example having any of the carbon sub-ranges noted above for C₁₋₂₀ alkyl. As used herein, “haloalkyl” refers to an alkyl group substituted with at least one halo group (e.g., Cl, Br, or I). A haloalkyl group can be perhalogenated. Unless otherwise indicated, a haloalkyl group can be an unsubstituted haloalkyl group or a substituted haloalkyl group.

In embodiments, the hydrophobic group includes C₂₋₂₀ alkenyl. As used herein, the term “alkenyl” is defined identically as “alkyl” except for containing at least one carbon-carbon double bond. The term Cn means the alkenyl group has “n” carbon atoms. For example, C₄ alkenyl refers to any alkenyl group that has 4 carbon atoms. C₂₋₂₀ alkenyl refers to an alkenyl group having a number of carbon atoms encompassing the entire range (i.e., 2 to 18 carbon atoms), as well as all subgroups (e.g., 2-17, 2-16, 3-18, 2, 3, 4, 5, 6, 7, 8, 9, 10, 11, 12, 13, 14, 15, 16, 17, 18, 19, and 20 carbon atoms). Nonlimiting examples of alkenyl groups include ethenyl, 1-propenyl, 2-propenyl, and butenyl. Unless otherwise indicated, an alkenyl group can be an unsubstituted alkenyl group or a substituted alkenyl group.

In embodiments, the hydrophobic group includes C₂₋₂₀ alkynyl. As used herein, the term “alkynyl” is defined identically as “alkyl” except for containing at least one carbon-carbon triple bond. The term Cn means the alkynyl group has “n” carbon atoms. For example, C₄ alkynyl refers to any alkynyl group that has 4 carbon atoms. C₂₋₂₀ alkynyl refers to an alkynyl group having a number of carbon atoms encompassing the entire range (i.e., 2 to 20 carbon atoms), as well as all subgroups (e.g., 2-17, 2-16, 3-18, 2, 3, 4, 5, 6, 7, 8, 9, 10, 11, 12, 13, 14, 15, 16, 17, 18, 19 and 20 carbon atoms). Nonlimiting examples of alkynyl groups include ethynyl, 1-propynyl, 2-propynyl, and butynyl. Unless otherwise indicated, an alkynyl group can be an unsubstituted alkynyl group or a substituted alkynyl group.

In embodiments, the hydrophobic group includes C₃₋₂₀ cycloalkyl. As used herein, the term “cycloalkyl” refers to a monocyclic or polycyclic hydrocarbon group containing three to twenty carbon atoms (e.g., 3, 4, 5, 6, 7, 8, 9, 10, 11, 12, 13, 14, 15, 16, 17, 18, 19, or 20 carbon atoms). The term Cn means the cycloalkyl group has “n” carbon atoms. For example, C₅ cycloalkyl refers to a cycloalkyl group that has 5 carbon atoms in the ring. C₅₋₈ cycloalkyl refers to cycloalkyl groups having a number of carbon atoms encompassing the entire range (i.e., 5 to 8 carbon atoms), as well as all subgroups (e.g., 5-6, 6-8, 7-8, 5-7, 5, 6, 7, and 8 carbon atoms). Nonlimiting examples of cycloalkyl groups include cyclopropyl, cyclobutyl, cyclopentyl, cyclohexyl, cycloheptyl, cyclooctyl, and adamantane. Unless otherwise indicated, a cycloalkyl group can be an unsubstituted cycloalkyl group or a substituted cycloalkyl group.

In embodiments, the hydrophobic group includes C₆₋₂₀ aryl. As used herein, the term “aryl” refers to monocyclic or polycyclic (e.g., fused bicyclic and fused tricyclic) carbocyclic aromatic ring systems. C₆₋₂₀ aryl indicates the number of carbon atoms in the aromatic ring system is 6-20 (e.g., at least 6, 8, 10, or 12 and/or up to 8, 10, 12, 14, 16, or 20). Nonlimiting examples of aryl groups include phenyl, naphthyl, tetrahydronaphthyl, phenanthrenyl, biphenylenyl, indanyl, indenyl, anthracenyl, fluorenyl, tetralinyl, and pyrene. Unless otherwise indicated, an aryl group can be an unsubstituted aryl group or a substituted aryl group.

In embodiments, the hydrophobic group includes C₁₋₂₀ heteroalkyl having 1-3 backbone heteroatoms selected from N, O, and S and the remainder backbone atoms being C, for example having any of the sub-ranges noted above for C₁₋₂₀ alkyl for it total number of backbone atoms. As used herein, the term “heteroalkyl” is defined similarly as alkyl, except the backbone of the alkyl chain contains one to three heteroatoms independently selected from oxygen, nitrogen, or sulfur, but the point of attachment of the heteroalkyl moiety is a carbon atom, not the heteroatom. Nonlimiting examples of heteroalkyl includes ethers, thioethers, amines, esters, thioesters, and amides. Unless otherwise indicated, an heteroalkyl group can be an unsubstituted heteroalkyl group or a substituted heteroalkyl group.

The hydrophobic group can optionally be substituted. For example, the hydrophobic group can be optionally substituted with other hydrophobic groups as described herein. Nonlimiting of suitable substituents include C₁₋₆ alkyl (e.g., C₂₋₆ alkyl, C₁₋₅alkyl, C₃₋₆ alkyl, etc.), C₁₋₆ haloalkyl (e.g., C₂₋₆ haloalkyl, C₁₋₅ haloalkyl, C₁₋₄haloalkyl, C₃₋₆ haloalkyl, etc.), C₃₋₁₀ cycloalkyl (e.g., C₃₋₆ cycloalkyl, C₃₋₅ cycloalkyl, C₃₋₈cycloalkyl, C₆₋₁₀cycloalkyl, etc.), and C₆₋₁₀ aryl (e.g., C₆₋₈ aryl, C₆aryl, C₁₀aryl, etc.).

In embodiments, the hydrophobic group includes one or more of n-hexyl, n-pentyl, n-butyl, phenyl, benzyl, biphenyl, pyrene, adamantane, and combinations thereof. In embodiments, the modified polysaccharide is modified with a single type of hydrophobic group (e.g., all hydroxyl groups of inulin that are modified are modified with n-hexyl). In embodiments, the modified polysaccharide includes two or more different hydrophobic groups (e.g., hydroxyl groups of inulin are modified with n-hexyl or n-pentyl). In embodiments, the polysaccharide is inulin modified with one or more selected from the group of n-hexyl, n-pentyl, n-butyl, phenyl, benzyl, and biphenyl.

As described herein, the modified polysaccharide can be free of charge. In embodiments, the modified polysaccharide is free of charge at a pH of about 2 to about 12, for example at a pH of at least about 2, 3, 4, 5, 6, 7, or 8 and/or up to a pH of about 6, 7, 8, 9, 10, 11, or 12, such as about 2 to about 12, about 2 to about 10, about 4 to about 10, about 6 to about 12, or about 6 to about 8.

The polysaccharide, prior to modification, can have a weight-average molecular weight (M_{w}) of about 1 kg/mol to about 30 kg/mol, for example at least about 1, 2, 5, 7, 10, 12, 15, 17, 19, 20, or 22 kg/mol and/or up to about 15, 17, 19, 20, 22, 25, 27, 29, or 30 kg/mol. In embodiments, the polysaccharide has a M_{w} of about 1 kg/mol to about 30 kg/mol, about 2 kg/mol to about 27 kg/mol, about 5 kg/mol to about 25 kg/mol, about 10 kg/mol to about 20 kg/mol, or about 14 kg/mol to about 16 kg/mol, prior to modification.

The modified polysaccharide (i.e., after modification with the hydrophobic group) can have a weight-average molecular weight (M_{w}) of about 1 kg/mol to about 50 kg/mol, for example at least about 1, 2, 5, 7, 10, 12, 15, 20, 25, 30, or 35 kg/mol and/or up to about 15, 20, 25, 30, 35, 37, 40, 45, or 50 kg/mol. In embodiments, the modified polysaccharide has a M_{w} of about 1 kg/mol to about 50 kg/mol, about 2 kg/mol to about 45 kg/mol, about 5 kg/mol to about 40 kg/mol, about 10 kg/mol to about 30 kg/mol, or about 20 kg/mol to about 50 kg/mol.

### Lipid Nanodiscs

The lipid nanodiscs of the disclosure generally include a lipid bilayer having a first hydrophilic face and a second hydrophilic face opposing the first hydrophilic face, and a hydrophobic edge between the opposing hydrophilic faces, and a modified polysaccharide of the disclosure encircling the hydrophobic edge of the lipid bilayer. The lipid is not particularly limited. The lipid can include a natural cell membrane extract. Suitable lipids include, but are not limited to phosphatidylethanolamines, phosphatidylcholines, phosphatidylglycerols, phosphatidylserines, cholesterols, sphingomyelin, gangliosides, lipopolysaccharides, phophatidylinositols, and derivatives of the foregoing. In embodiments, the lipid comprises at least one of phosphatidylethanolamines, phosphatidylcholines, phosphatidylglycerols, phosphatidylserines, cholesterols, sphingomyelin, gangliosides, lipopolysaccharides, and phophatidylinositols. In embodiments, the lipid is a phospholipid. In embodiments, the phospholipid includes a phosphatidylcholine. In embodiments, the lipid includes dipalmitoylphosphatidylcholine (DMPC)

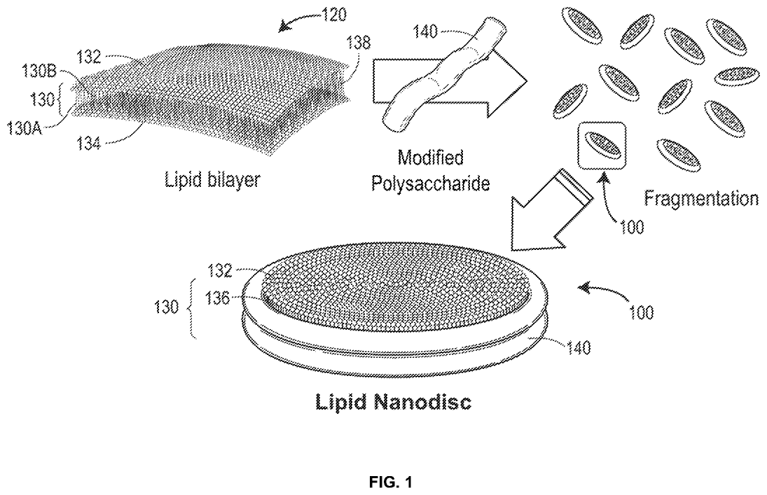

FIG. 1 illustrates a lipid nanodisc 100 according to the disclosure, which includes a lipid 120 and a modified polysaccharide 140. The lipid 120 in the nanodisc 100 forms a lipid bilayer 130 including a first hydrophilic face 132 and a second hydrophilic face 134, wherein the first hydrophilic face 132 and the second hydrophilic face 134 are opposing, and a hydrophobic edge 136 between the opposing hydrophilic faces 132, 134. The hydrophobic edge 136 is made up of the hydrophobic tails 138 from both layers of the lipid bilayer 130. The center of the hydrophobic edge 136 is the point at which the hydrophobic tail 138 from one layer 130A of the bilayer 130 meets the hydrophobic tail 138 from the second layer 130B of the bilayer 130. The nanodisc 100 further includes a modified polysaccharide 140 of the disclosure encircling the hydrophobic edge 136 of the lipid bilayer 130.

The lipid and the polysaccharide can be present in the lipid nanodiscs in a weight ratio of about 1:0.1 to about 1:20, for example at least about 1:0.01, 1:0.1, 1:0.5, 1:1, 1:2, 1:5, 1:7, 1:10, 1:12, or 1:15, and/or up to about 1:5, 1:8, 1:10, 1:12, 1:15, 1:17, 1:18, or 1:20. In embodiments, the lipid and the polysaccharide are present in the lipid nanodiscs in a weight ratio of about 1:0.01 to about 1:20, about 1:0.1 to about 1:19, about 1:0.5 to about 1:18, about 1:1 to about 1:15, about 1:2 to about 1:12, about 1:5 to about 1:10, or about 1:6 to about 1:8.

The lipid nanodiscs of the disclosure can have a diameter in a range of about 6 nm to about 100 nm, for example, about 6 nm to about 100 nm, about 10 nm to about 90 nm, about 20 nm to about 90 nm, about 30 nm to about 80 nm, about 40 nm to about 80 nm, about 50 nm to about 70 nm, or about 55 nm to about 65 nm. In some embodiments, the nanodisc has a diameter less than or equal to 40 nm, for example, in a range of about 6 nm to 40 nm, about 10 nm to about 35 nm, about 6 nm to about 20 nm, about 20 nm to about 35 nm, or about 25 nm to about 30 nm. In some embodiments, the nanodisc has a diameter greater than 40 nm, for example, 41 nm to about 100 nm, about 45 nm to about 90 nm, about 50 nm to about 80 nm, about 50 nm to about 70 nm, or about 60 nm. In some embodiments, the nanodisc has a diameter less than or equal to 20 nm, for example, in a range of about 6 nm to 20 nm, about 8 nm to about 18 nm, about 10 nm to about 16 nm, or about 12 nm to about 14 nm. Nanodiscs having a diameter greater than 20 nm can be referred to as “macro-nanodiscs.” The size of the nanodisc can be controlled by changing the lipid:polysaccharide weight ratio during preparation. In general, as the amount of modified polysaccharide increases relative to the amount of lipid, the size of the resulting nanodisc decreases. Similarly, as the amount of modified polysaccharide decreases relative to the amount of lipid, the size of the resulting nanodisc increases.

In some embodiments, the lipid nanodisc can be characterized in that when a magnetic field is applied, the nanodisc aligns with the magnetic field. Such a characteristic can be advantageous, for example, when characterizing the nanodisc (or a membrane protein provided therein) by NMR spectroscopy. The nanodiscs can align such that the lipid bilayer is normal-oriented perpendicular to the direction of the applied magnetic field.

The lipid nanodisc can further include a membrane protein. The membrane protein can be any protein that interacts with or is part of a biological membrane, and can be permanently anchored or temporarily anchored to a lipid bilayer. Suitable membrane proteins include, but are not limited to U-15N Cytb5, cytochromes such as cytochrome b5, cytochrome P450, cytochrome P450 reductase, cytochrome c, outer membrane proteins or integral outer membrane proteins, photosystem II, voltage-gated ion channels, beta barrel, and G-protein-coupled receptors (GPCRs). When a membrane protein is included in the lipid nanodisc, the membrane protein spans across at least one half of the lipid bilayer, from one hydrophilic face to the center of the hydrophobic edge (i.e., the point at which the hydrophobic tail 138 from one layer 130A of the bilayer 130 meets the hydrophobic tail 138 from the second layer 130B of the bilayer 130). In some embodiments, the membrane protein spans across the entire lipid bilayer from the first hydrophilic face to the second hydrophilic face at least once. In some embodiments, the membrane protein spans across the entire lipid bilayer from the first hydrophilic face to the second hydrophilic face more than once.

In embodiments, the lipid nanodisc further includes a drug and/or a vaccine. The drug and/or vaccine can be incorporated in, encapsulated by, or entangled with the lipid nanodisc. When a drug and/or vaccine is present in the lipid nanodisc, the lipid nanodisc can act as a carrier and/or delivery system. In general, the lipid nanodisc can be used as a carrier for any hydrophobic and/or low solubility drug and/or vaccine. Suitable drugs include, but are not limited to, those classified as Class II and IV according to the Biopharmaceutical Classification System (BCS). BCS Class II drugs have high permeability and low solubility, and BCS Class IV drugs have low permeability and low solubility. These drugs generally must be administered in a formulation to increase their solubility and bioavailability. Examples of suitable drugs include, but are not limited to, benzylpenicillin, bicalutamide, bifonazole, glibenclamide, ezetimibe, and aceclofenac. Suitable vaccines include, but are not limited to, vaccines based on peptides, proteins, RNA, DNA, their functionalized variants (e.g., cholesterol/lipid modified DNA or RNA), other chemical entities, or a combination of the foregoing. In embodiments, the vaccine comprises a vaccine based on peptides, proteins, RNA, DNA, functionalized variants of the foregoing, and combinations of any of the foregoing. In embodiments, the vaccine comprises a vaccine based on RNA, DNA, functionalized variants of the foregoing, or any combination of the foregoing. In embodiments, the vaccine comprises a vaccine based on RNA, functionalized variants of RNA, or a combination of the foregoing. As used herein and unless specified otherwise, the term “RNA” encompasses mRNA, tRNA, rRNA, snRNA and other non-coding RNAs.

The nanodisc can be stable at a variety of pH levels, as well as in the presence of ions. For example, the nanodisc can be stable at a pH ranging from about 2 to about 12, for example at least about 2, 3, 4, 5, 6, 7, or 8 and/or up to about 7, 8, 9, 10, 11, or 12. In embodiments, the lipid nanodisc is stable at a pH level of about 2 to about 12, about 3 to about 10, about 4 to about 8, or about 5 to about 6. The nanodisc can be stable in the presence of divalent metal ions. For example, the nanodisc can be stable in the presence of up to about 150 mM of a divalent metal ion, such as up to about 150, 125, 100, 90, 80, 70, 60, 50, 40, 30, 20, 10, or 5 mM of a divalent metal ion, such as Ca²⁺, Mg²⁺, Cu²⁺, and Zn²⁺. In embodiments, the nanodisc is stable in the presence of 100 mM of Ca²⁺.

The lipid nanodisc can be characterized by a solubilization efficiency of at least 50%, for example at least 50, 55, 60, 65, 70, 75, or 80% and/or up to 70, 75, 85, 90, 95, 99, or 100%. In embodiments, the nanodisc is characterized by a solubilization efficiency of about 50% to about 100%, about 60% to about 100%, about 70% to about 100% or about 80% to about 100%. Solubilization efficiency refers to the ability of the modified polysaccharide to solubilize a hydrophobic and/or low solubility drug and/or vaccine, or a membrane protein from the cellular environment (such as cell lysate) by forming nanodiscs with the protein along with the surrounding lipids. The solubilization efficiency of a membrane protein can be obtained by measuring the soluble membrane protein concentration using a suitable assay, such as a Pierce™ BCA Protein Assay. The obtained concentrations can be normalized to a known detergent, such as n-dodecyl-B-D-maltoside (DDM). The solubilization efficiency of a hydrophobic and/or low solubility drug and/or vaccine can be obtained by comparing the amount or concentration of residual drug and/or vaccine after nanodisc formation to the initial amount or concentration of drug and/or vaccine available to be loaded prior to nanodisc formation.

### Method of Preparing Lipid Nanodisc

Further provided herein are methods of making a lipid nanodisc 100. In embodiments, the method includes contacting a lipid 120 and modified polysaccharide 140 (FIG. 1). Advantageously, the nanodiscs of the disclosure are easy to prepare, inexpensive, and stable for up to a month at 4° C. When stored at lower temperatures, e.g., −80° C., the nanodiscs of the disclosure can be stable for up to 6 months. Furthermore, the nanodiscs of the disclosure can advantageously be characterized by common biophysical techniques, such as circular dichroism (CD), UV/Vis and fluorescence spectroscopy.

The lipid and modified polysaccharide can be any lipid and modified polysaccharide described herein. The method of preparing the lipid nanodisc includes contacting the lipid and the modified polysaccharide. In embodiments, the lipid is provided as a multilamellar vesicle. Without intending to be bound by theory, it is believed that when the lipid is provided as a multilamellar vesicle, the modified polysaccharide can be inserted into the lipid bilayer and break the multilamellar vesicle into nanodisc shaped lipoparticles. The lipid can include a natural cell membrane extract.

The lipid can further include a membrane protein such that the resulting lipid nanodisc includes a membrane protein spanning across at least one half of the lipid bilayer from one hydrophilic face to the center of the hydrophobic edge (i.e., the point at which the hydrophobic tail 138 from one layer 130A of the bilayer 130 meets the hydrophobic tail 138 from the second layer 130B of the bilayer 130). In embodiments, the lipid includes a membrane protein such that the resulting lipid nanodisc includes a membrane protein spanning across the entire lipid bilayer from one hydrophilic face to the second hydrophilic face at least once. In some embodiments, the membrane protein spans across the entire lipid bilayer from the first hydrophilic face to the second hydrophilic face more than once.

The contacting step can include admixing the lipid and the modified polysaccharide in solution. An aqueous solution of modified polysaccharide can be prepared prior to contacting the modified polysaccharide with the lipid. A lipid dispersion can be prepared prior to contacting the modified polysaccharide with the lipid. The contacting step can include admixing the modified polysaccharide solution and the lipid dispersion. The solutions, suspensions, and dispersions of the disclosure can be substantially free of a detergent. As used herein, “substantially free” means that the solution and/or suspension does not contain significant amounts of a purposefully added detergent. Thus, incidental or background quantity of detergents (e.g., less than about 100 ppb) can be present in the solution and/or suspension and be within the scope of the disclosure.

The contacting step can optionally further include providing a buffer to regulate the pH of the solution. The contacting step can be carried out at any suitable pH in which the modified polysaccharide is stable and soluble, for example, in a range of about 0 to about 14, about 1 to about 12, about 2 to about 11, about 2 to about 10, about 2.5 to about 10, about 3 to about 8, about 7 to about 12, or about 3 to about 9, for example, about 2.5, about 3, about 3.5, about 4, about 4.5, about 5, about 5.5, about 6, about 6.5, about 7, about 7.5, about 8, about 8.5, about 9, about 9.5, or about 10.

The weight ratio of the lipid to the modified polysaccharide of the disclosure can be provided in a ratio of about 1:0.1 to about 1:20, for example at least about 1:0.01, 1:0.1, 1:0.5, 1:1, 1:2, 1:5, 1:7, 1:10, 1:12, or 1:15, and/or up to about 1:5, 1:8, 1:10, 1:12, 1:15, 1:17, 1:18, or 1:20. In embodiments, the lipid and the polysaccharide are present in the lipid nanodiscs in a weight ratio of about 1:0.01 to about 1:20, about 1:0.1 to about 1:19, about 1:0.5 to about 1:18, about 1:1 to about 1:15, about 1:2 to about 1:12, about 1:5 to about 1:10, or about 1:6 to about 1:8. In general, as the weight of the modified polysaccharide is increased relative to the weight of the lipid, the maximum diameter of the resulting nanodiscs decrease. In embodiments, there is an asymptotic value for the relative amount of the modified polysaccharide above which there is no further substantial decrease in the size/diameter of the nanodisc.

The contacting step can optionally further include providing a drug and/or a vaccine. The amount of drug and/or vaccine provided in the contacting step is not particularly limited. The amount of drug and/or vaccine can be selected based on the desired amount of drug and/or vaccine to be provided in the nanodisc and the solubilization efficiency for the drug and/or vaccine.

The formation of the lipid nanodiscs of the disclosure can be confirmed and characterized using a number of well-known techniques such as static light scattering (SLS), dynamic light scattering (DLS), size-exclusion chromatography (SEC), Fourier-transform infrared spectroscopy (FT-IR), solid-state nuclear magnetic resonance (ssNMR), and transmission electron microscopy (TEM). Advantageously, when the nanodiscs are less than or equal to about 20 nm, the structure of the nanodiscs can be determined based on solution NMR techniques and when the nanodiscs are greater than about 20 nm, the nanodiscs can be magnetically-aligned which is advantageous for solid-state NMR studies. Furthermore, as the modified polysaccharides of the disclosure can be free of styrene, the nanodiscs can be characterized using biophysical techniques such as circular dichroism (CD), ultraviolet-visible spectroscopy (UV/Vis), and fluorescence spectroscopy.

### Method of Characterizing Membrane Proteins

The disclosure further provides a method of characterizing a membrane protein, the method including contacting a lipid nanodisc of the disclosure with a membrane protein to form a membrane protein-nanodisc including the membrane protein spanning across the lipid bilayer from the first hydrophilic face, to the second hydrophilic face of the lipid nanodisc and characterizing the lipid nanodisc including the membrane protein. In embodiments, the membrane protein spans across the entire lipid bilayer from the first hydrophilic face to the second hydrophilic face more than once. The membrane protein can be any membrane protein disclosed herein. In embodiments, the membrane protein spans across half of the lipid bilayer from one hydrophilic face to the center of the hydrophobic edge.

In embodiments, the contact includes admixing the lipid nanodisc and membrane protein in solution. In embodiments, the solution is substantially free of detergent.

Characterization can include at least one of a structural characterization of the membrane protein or a functional characterization of the membrane protein. Suitable membrane protein characterization methods include solution and solid state nuclear magnetic resonance (NMR), circular dichroism (CD), electron paramagnetic resonance (EPR), Fourier transform infrared spectroscopy (FTIR), resonance Raman spectroscopy, ultraviolet-visible spectroscopy (UV/vis), cryo-electron microscopy (cryo-EM), surface plasmon Raman spectroscopy, sum frequency generation (SFG), fluorescence, including single molecule fluorescence and coherent anti-Stokes Raman (CARS), small angle x-ray scattering (SAXS), scanning electron microscopy (SEM), atomic force microscopy (AFM) and enzymatic assays Membrane protein structure and dynamics can be characterized using NMR techniques. For example, membrane protein-nanodiscs having a diameter of about 20 nm or less can be characterized using solution NMR and membrane protein-nanodiscs having a diameter greater than about 20 nm can be characterized using solid state NMR. Advantageously, the nanodiscs of the disclosure can include additional features for enhancing characterization by NMR, for example, the nanodisc can be characterized in that when a magnetic field is applied, the nanodisc aligns with the magnetic field and the nanodisc optionally includes a chelating group having a metal ion bound thereto which allows paramagnetic resonance characterization.

Magnetically aligned nanodiscs can provide a novel membrane mimetic environment for the structural investigation of several membrane proteins by measuring 1H-15N heteronuclear dipolar couplings. One of the most popular approaches to measure heteronuclear dipolar couplings in ssNMR is the 2D separation of heteronuclear dipolar interactions according to chemical shifts. This class of experiments is known as Separated Local Field (SLF) spectroscopy. Polarization Inversion and Spin Exchange at Magic Angle (PISEMA) is a well-known and useful NMR technique for structural studies of a variety of biological systems.

The modified polysaccharide-based lipid nanodiscs of the disclosure can be advantageous for one or more applications including, reconstitution of membrane proteins, purification of membrane proteins or peptides and their complexes with other soluble or membrane-bound proteins, DNA, RNA, drugs, ligands, and/or other chemicals, drug delivery, vaccine delivery, as an adjuvant in vaccine delivery, and/or controlling the aggregation of amyloid peptides or proteins.

The above described aspects and embodiments can be better understood in light of the following examples, which are merely intended to be illustrative and are not meant to limit the scope in any way.

### EXAMPLES

### Example 1—Synthesis and Characterization of Modified Polysaccharide

A modified inulin was synthesized and characterized in accordance with General Scheme 1, below.

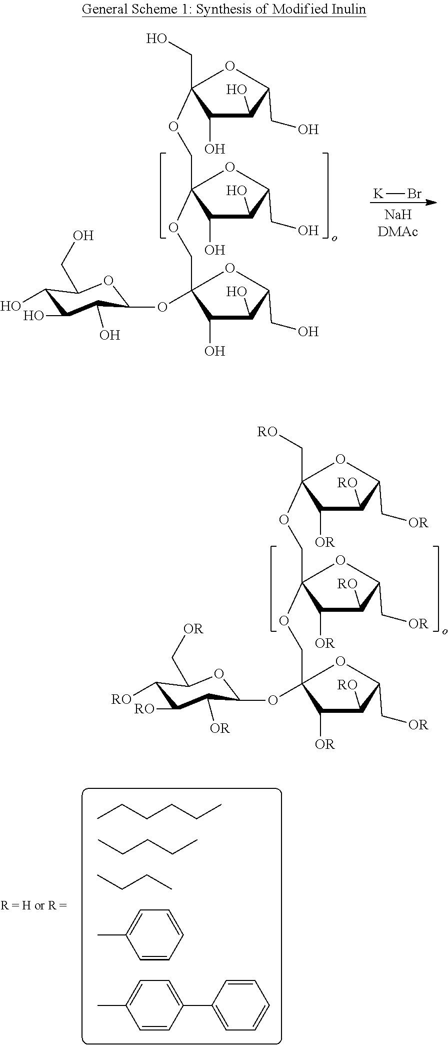

Inulin extracted from chicory was purchased from SIGMA-ALDRICH and was used without purification. It was reacted with alkylbromide (e.g., n-hexyl bromide, n-pentyl bromide, n-butyl bromide), benzyl bromide, and biphenyl-bromide (collectively, “R—Br”) in the presence NaH and dimethylacetamide (DMAc) to produce an alkylated inulin derivative. The extent of functionalization was controlled by the relative concentrations of inulin and R—Br. The product was characterized using solution NMR and MALDI-mass experiments. The MALDI-mass-spec data of the starting material showed an average degree of polymerization of 14.

A ¹H NMR spectrum of a hexyl-derivative of inulin was obtained. Tetramethylsilane (TMS) was used as reference and set at 0 ppm. The peak from H₁-Glc appearing at 5.4 ppm was used as a reference, and the integrated area of the peak was set to 1 proton. The extent of functionalization of the terminal methyl group of the hexyl group at 0.9 ppm was determined using the NMR peak. A 2D ¹H/¹³C NMR experiment correlating the chemical shifts of ¹H and ¹³C nuclei was performed to further confirm the product. The 2D ¹H/¹³C NMR spectrum showed the peaks from the hexyl, as well as the peaks from the fructose repeating units of inulin. The 2D spectrum revealed the random functionalization of the R group in the primary (i.e. —CH2-OH) as well as in the secondary (ring —OH) —OH groups of inulin.

Carbon-13 cross-polarization and magic angle spinning (CPMAS) experiments were carried out to further characterize the starting material and the products. The change in the ¹³C chemical shift observed for the derivatives suggested the random functionalization of —OH groups which corresponded to the ¹H solution NMR.

### Example 2—Preparation of Lipid Nanodiscs

Modified inulin derivative stock solutions were prepared by dissolving 10 mg of the modified polysaccharide in 1 mL of appropriate buffer. Lipid stock solutions were prepared by the addition of 10 mg of dipalmitoylphosphatidylcholine (DMPC) powder to 1 mL of buffer and subjected to three freeze-thaw cycles until a homogenous dispersion of lipid was obtained. Lipid nanodiscs were prepared by adding the polymer stock solution to the lipid solution in various ratios. The resulting solutions were incubated overnight at 4° C. on an orbital shaker. The resulting nanodiscs were subjected to size exclusion chromatography, if needed. The resulting nanodiscs were characterized using dynamic light scattering (DLS) and transmission electron microscopy (TEM).

Images of the nanodiscs formed by the modified inulin derivatives were obtained via TEM. The TEM image for DMPC nanodiscs formed by benzyl-modified inulin is shown in FIG. 3. The TEM image revealed nanodiscs having a diameter of about 25±5 nm.

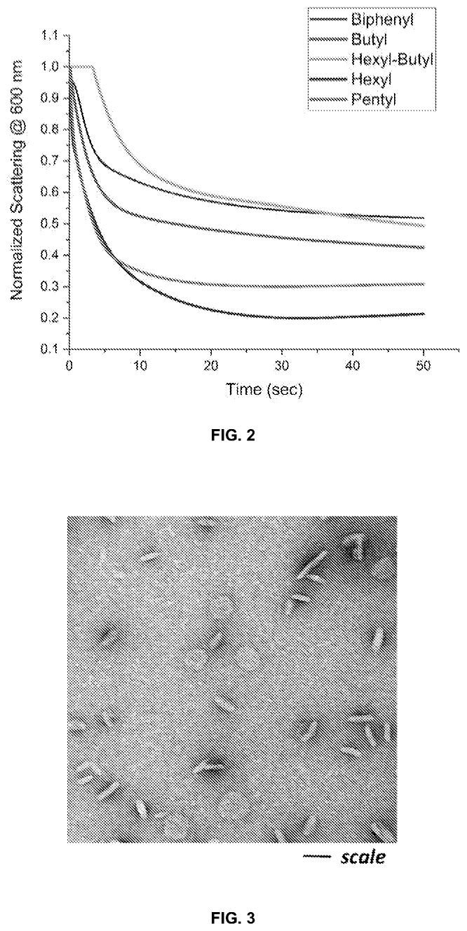

Equal volumes of DMPC liposomes and polymer stock solutions were injected into a stopped flow cell and the static light scattering intensity was monitored using a photomultiplier at a right angle. Each measurement was repeated three times and the average of three spectral traces were plotted in FIG. 2. The decreasing static light scattering profiles suggested that the solubilization capabilities of the derivatives with hexyl and pentyl hydrophobic groups exhibited faster kinetics and the maximum solubilization effects.

### Example 3—Characterization of Lipid Nanodiscs

The stability of the lipid nanodiscs formed by modified inulin derivatives was evaluated at various pH levels and in the presence of divalent metal ions.

The nanodiscs were prepared by the addition of the modified polysaccharide to DMPC liposomes and diluted to give identical concentrations of lipids. Different pH samples were obtained by the addition of 1M HCl and/or 1M NaOH. Metal ion concentration was adjusted by the addition of 1M CaCl₂).

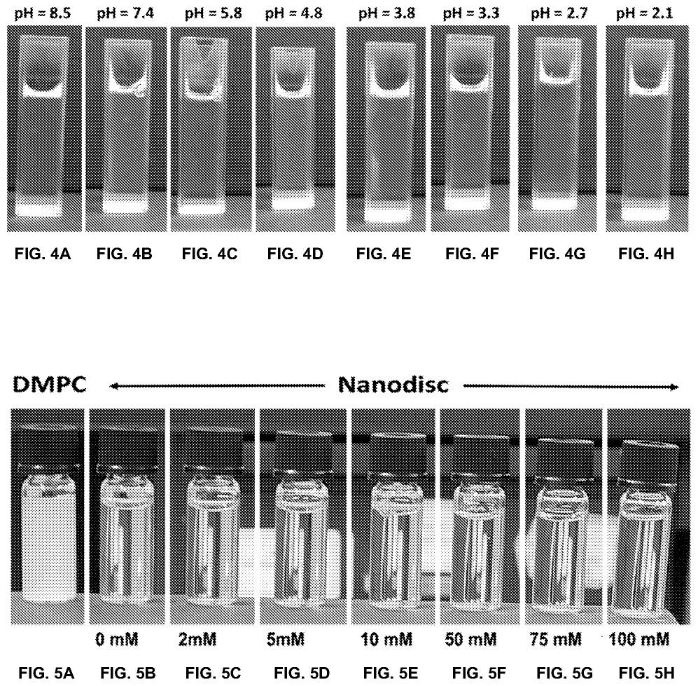

FIGS. 4A-4F show images of the DMPC nanodiscs formed from pentyl-modified inulin derivatives at various pH levels. As shown in the images, there was no visual degradation of the nanodiscs at any of the pH levels evaluated as seen for DMPC liposomes alone without the modified polysaccharide (FIG. 5A).

FIGS. 5B-5H show images of the DMPC nanodiscs formed from pentyl-modified inulin derivatives in solution with various concentrations of calcium ions (Ca²⁺). As shown in the images, there was no visual degradation of the nanodiscs at any of the concentrations evaluated as seen for DMPC liposomes alone without the modified polysaccharide (FIG. 5A).

Notably, the pentyl-modified inulin derivatives, alone, were not stable against pH changes of in the presence of calcium ions.

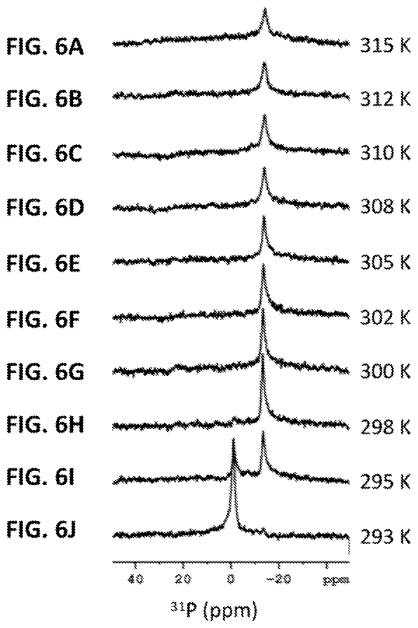

The lipid nanodiscs were further characterized by their magnetic alignment. Phosphorus-31 (³¹P) NMR experiments were performed to characterize the ability of DMPC-pentyl-modified inulin nanodiscs to align in the presence of an external magnetic field. The spectra of these experiments are shown in FIGS. 6A-6J. A reference peak from H₃PO₄ was set at 0 ppm. As shown in FIGS. 6A-6J, the nanodiscs were isotropic below the main phase transition of DMPC (i.e., <293 K) as characterized by a peak at about 1 ppm. On the other hand, above the main phase transition of DMPC (i.e., >295 K), the appearance of a peak at about −15 ppm suggested that the nanodiscs were aligned with the lipid-bilayer-normal oriented perpendicular to the direction of the external magnetic field.

### Example 4—Solubilization of E. coli Cell Membranes

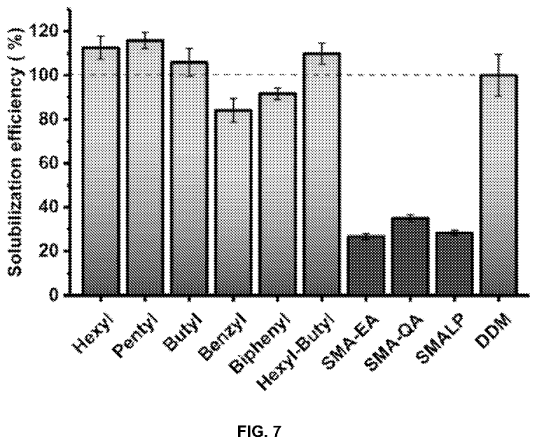

The modified inulin derivatives were added to E. coli cell lysates. The membrane-containing soluble fraction was separated by centrifugation. The estimated extracted membrane protein content was obtained using a Pierce BCA protein assay kit (THERMOFISCHER SCIENTIFIC). As shown in FIG. 7, the estimated membrane protein content was plotted with reference to the result obtained using a detergent (non-ionic N-dodecyl-beta-maltoside; DDM) (set at 100%). The abilities of different types nanodisc-forming polysaccharides to extract membrane proteins were compared.

The results demonstrated that lipid nanodiscs prepared using the modified polysaccharides of the disclosure performed significantly better compared to those prepared from other polymers such as styrene-maleic acid (SMA) and its derivatives (SMA-QA=SMA with quaternary ammonium groups; SMA-EA=SMA with ethanolamine groups; and SMALP=styrene maleic acid lipid particles).

### REFERENCES

1. Overington, J. P.; Al-Lazikani, B.; Hopkins, A. L., How many drug targets are there? Nature Reviews Drug Discovery 2006, 5 (12), 993-996.
2. Denisov, I. G.; Sligar, S. G., Nanodiscs for structural and functional studies of membrane proteins Nat Struct Mol Biol 2016, 23 (6), 481-6.
3. Lee, S. C.; Knowles, T. J.; Postis, V. L.; Jamshad, M.; Parslow, R. A.; Lin, Y. P.; Goldman, A.; Sridhar, P.; Overduin, M.; Muench, S. P.; Dafforn, T. R., A method for detergent-free isolation of membrane proteins in their local lipid environment. Nat Protoc 2016, 11 (7), 1149-62.
4. Ravula, T.; Hardin, N. Z.; Ramadugu, S. K.; Cox, S. J.; Ramamoorthy, A., Formation of pH-Resistant Monodispersed Polymer-Lipid Nanodiscs. Angew Chem Int Ed Engl 2018, 57 (5), 1342-1345.
5. Ravula, T.; Ramadugu, S. K.; Di Mauro, G.; Ramamoorthy, A., Bioinspired, Size-Tunable Self-Assembly of Polymer-Lipid Bilayer Nanodiscs. Angew Chem Int Ed Engl 2017, 56 (38), 11466-11470.
6. Yasuhara, K.; Arakida, J.; Ravula, 1.; Ramadugu, S. K.; Sahoo, B.; Kikuchi, J. I.; Ramamoorthy, A., Spontaneous Lipid Nanodisc Fomation by Amphiphilic Polymethacrylate Copolymers. J Am Chem Soc 2017, 139 (51), 18657-18663.
7. Hardin, N. Z.; Ravula, 1.; Mauro, G. D.; Ramamoorthy, A., Hydrophobic Functionalization of Polyacrylic Acid as a Versatile Platform for the Development of Polymer Lipid Nanodisks. Small 2019, 15 (9), e1804813.
8. Oluwole, A. O.; Danielczak, B.; Meister, A.; Babalola, J. O.; Vargas, C.; Keller, S., Solubilization of Membrane Proteins into Functional Lipid-Bilayer Nanodiscs Using a Diisobutylene/Maleic Acid Copolymer. Angew Chem Int Ed Engl 2017, 56 (7), 1919-1924.
9. Ravula, 1.; Hardin, N. Z.; Bai, J.; Im, S. C.; Waskell, L.; Ramamoorthy, A., Effect of polymer charge on functional reconstitution of membrane proteins in polymer nanodiscs. Chem Commun (Camb) 2018, 54 (69), 9615-9618.

1. A lipid nanodisc, comprising:
a lipid bilayer comprising a first hydrophilic face and a second hydrophilic face opposing the first hydrophilic face, and a hydrophobic edge between the opposing hydrophilic faces; and,
a polysaccharide encircling the hydrophobic edge of the lipid bilayer, wherein the polysaccharide is modified with a hydrophobic group.

2. The lipid nanodisc of claim 1, wherein the polysaccharide comprises a linear polysaccharide having a degree of polymerization of about 100 or less.

3. The lipid nanodisc of claim 1, wherein the polysaccharide is one or more selected from the group of inulin, cellulose, amylose, and any derivative of the foregoing.

4. The lipid nanodisc of claim 1, wherein the hydrophobic group is C₁₋₂₀ alkyl, C₁₋₂₀ haloalkyl, C₂₋₂₀ alkenyl, C₂₋₂₀ alkynyl, C₃₋₂₀cycloalkyl, C₆₋₂₀ aryl, or C₁₋₂₀ heteroalkyl having 1-3 backbone heteroatoms selected from N, O, and S.

5. (canceled)

6. (canceled)

7. The lipid nanodisc of claim 1, wherein the polysaccharide is inulin modified with one or more selected from the group of n-hexyl, n-pentyl, n-butyl, phenyl, benzyl, and biphenyl.

8. The lipid nanodisc of claim 1, wherein the polysaccharide is free of amine.

9. The lipid nanodisc of claim 1, wherein the hydrophobic group is bound to the polysaccharide via a linking group selected from the group consisting of an ether group, an ester group, and an amide group.

10. The lipid nanodisc of claim 1, wherein about 5% to about 50% of pendant oxygen atoms on the polysaccharide are modified with a hydrophobic group.

11. The lipid nanodisc of claim 1, wherein the polysaccharide is free of charge at a pH of about 2 to about 12.

12. (canceled)

13. The lipid nanodisc of claim 1, wherein the polysaccharide has a weight-average molecular weight (M_{w}) of about 1 kg/mol to about 30 kg/mol.

14. The lipid nanodisc of claim 1, wherein the lipid comprises at least one of phosphatidylethanolamines, phosphatidylcholines, phosphatidylglycerols, phosphatidylserines, cholesterols, sphingomyelin, gangliosides, lipopolysaccharides, phosphatidylinositols, and derivatives of the foregoing.

15. (canceled)

16. The lipid nanodisc of claim 1, wherein the lipid comprises a natural cell membrane extract.

17. The lipid nanodisc of claim 1, wherein the lipid and the polysaccharide are present in a weight ratio of about 1:0.1 to about 1:20.

18. The lipid nanodisc of claim 1, wherein the nanodisc has a diameter in a range of about 6 nm to about 100 nm.

19. (canceled)

20. The lipid nanodisc of claim 1, wherein the nanodisc is stable in the presence of up to about 150 mM of a divalent metal ion.

21. (canceled)

22. The lipid nanodisc of claim 1, further comprising a membrane protein spanning across the lipid bilayer from the first hydrophilic face to the second hydrophilic face.

23. (canceled)

24. (canceled)

25. The lipid nanodisc of claim 1, further comprising a drug, a vaccine, or a combination thereof.

26. The lipid nanodisc of claim 25, wherein the vaccine comprises a vaccine based on peptides, proteins, RNA, DNA, functionalized variants of the foregoing, or a combination of any one of the foregoing.

27. (canceled)

28. A method of characterizing a membrane protein, the method comprising:
contacting the lipid nanodisc of any one of the preceding claims with a membrane protein to form a membrane protein-nanodisc comprising the membrane protein spanning across the lipid bilayer from the first hydrophilic face to the second hydrophilic face; and,
characterizing the lipid nanodisc comprising the membrane protein.

29.-32. (canceled)

33. A method of preparing the lipid nanodisc of claim 1, the method comprising contacting the lipid and the polysaccharide to form the lipid nanodisc.

34.-46. (canceled)
